(19) United States
(12) Patent Application Publication
(10) Pub. No.: US 2025/0263353 A1
(43) Pub. Date: Aug. 21, 2025

(54) RENEWABLE ETHYLENE AT MINIMUM CARBON INTENSITY

(71) Applicant: Chevron U.S.A. Inc., San Ramon, CA (US)

(72) Inventors: Theodorus Ludovicus Michael MAESEN, Spring, TX (US); Cong-Yan CHEN, Kensington, CA (US); Joel E. SCHMIDT, Oakland, CA (US)

(73) Assignee: Chevron U.S.A. Inc., San Ramon, CA (US)

(21) Appl. No.: 19/060,018

(22) Filed: Feb. 21, 2025

Related U.S. Application Data
(60) Provisional application No. 63/556,155, filed on Feb. 21, 2024.

(51) Int. Cl. C07C 4/04; C10G 1/02; C10G 9/36

(52) U.S. Cl. CPC C07C 4/04; C10G 1/02; C10G 9/36; C10G 2300/1011; C10G 2400/20

(57) ABSTRACT

Provided is a stable and efficient process for preparing ethylene in improved yields when starting with a bio feedstock. The process first involves a hydrodeoxygenation reaction in which n-paraffins are made. The n-paraffins are then subjected to a hydroconversion reaction. The hydroconversion reaction is run in the presence of an LTA type zeolite, which zeolite has an acid site concentration preferably about 2.7 mol/l or greater. A boiling range of n-paraffins is then collected from the hydroprocessing reactor comprising C₂-C₆ n-paraffins. The collected C₂-C₆ n-paraffins can then be pyrolyzed in a steam cracker with good results including improved ethylene production.

### CROSS REFERENCE TO RELATED APPLICATIONS

The present application claim priority to U.S. Patent Application No. 63/556,155, filed Feb. 21, 2024, the complete disclosure of which is incorporated herein by reference in its entirety.

### TECHNICAL FIELD

Stable process for hydroconverting normal paraffins obtained from a renewable source into lighter normal paraffins with minimal formation of iso-paraffins. The process hydro-normalizes long (C₁₀⁺) n-paraffins obtained from renewable bio feedstocks. This thereby tailors a suitable feed stream for naphtha steam crackers.

### BACKGROUND

Steam crackers can be distinguished based on feed type. Where ethane is inexpensive (currently in the US and in the Middle East) most gas crackers are designed to pyrolyze mostly ethane. More typically, gas crackers can handle feedstocks including liquefied petroleum gas (LPG). Liquid crackers tend to focus on naphtha. Mixed feed stream crackers have the flexibility to crack both gas and liquid feeds, and some can handle feedstocks with an end boiling point significantly higher than 380° F., a typical end point of naphtha. Some mixed feed steam crackers were commercialized with the ability to pyrolyze feedstocks efficiently with an end boiling point as high as 900° F. viz. by including a Heavy Oil Processing System or HOPS. However, most commercial liquid steam crackers are referred to as “naphtha crackers” because they are limited to feedstocks in the naphtha boiling range (90-380° F.). Such naphtha crackers are particularly prevalent in geographies where ethane is in short supply (currently in Asia, Europe, Africa, Australia).

Previously, both UOP and ExxonMobil have commercialized n-paraffin pressure swing adsorption processes (e.g., Molex™, IsoSiv™,) to concentrate the n-paraffins from light naphtha to enable subsequent octane upgrading through isomerization or to enhance the ethylene yield in subsequent steam cracking. Extension of these processes beyond pentane and hexane through Maxene™, and Ensorb™ processes has was limited as a result of added operational and energy costs of such a concentration process. These costs are quite high because the desorption of n-paraffins requires excess energy when n-paraffins become much longer than n-heptane and because thermodynamics disfavors isomerization of iso-paraffins into n-paraffins—it favors the reaction into the opposite direction.

Revamping a steam cracker to enable higher boiling feed components is expensive. Thus, lowering the feed boiling point to a level that existing steam crackers can handle without degrading the feedstock by forming isomers becomes the most practical objective. This is difficult when dealing with a heavy feed having long n-paraffins, e.g., C₁₂⁺ in length.

For over a century, hydrocracking and catalytic cracking have been used to reduce the boiling point and to increase the H/C ratio of crude oils and coal-derived liquids in refineries. Inherently, these catalytic processes also increased the iso-paraffin content of the lighter fractions. This was desirable when the intended use of the cracking products was combustion in an internal combustion engine. However, crude oil is now increasingly used for chemical manufacture, and high iso-paraffin content has become an undesirable side effect of acid-catalyzed (hydro) cracking.

There is an urgent need in the industry to diversify the crude oil value chain. The preparation of n-paraffins as a steam cracker feedstock has become important. Minimal formation of iso-paraffins is important. The industry would welcome a stable and efficient catalytic process for hydroconverting normal paraffins into lighter normal paraffins with minimal formation of iso-paraffins. The stability of such a process would create great efficiency and economic value by increasing carbon efficiency through minimizing the methane and pyrolysis oil make associated with steam cracking iso-instead of n-paraffins.

There is also a growing end user demand for sustainable, bio-originating and possibly recycled alternatives in the field of chemicals, as well as fuels. Although not yet mandated to contain bio-originating products, there are clear signs of legislative directives emerging also for these areas. Currently, there is a limited offering of bio-originating alternatives available for the above-mentioned applications. Further the bio-originating alternatives are typically not cost competitive with the conventional offering, which has limited the development of the bio-originating chemicals and fuels. Olefins such as ethylene, propylene, and butylene are the raw materials of polyolefins. Polyolefins are one of the key building blocks of the global economy. The demand is growing. Providing such chemicals from bio feedstocks would be of great value to the industry and to the environment. And providing a process that can satisfy both of these urgent needs would be of particular value to the industry.

### SUMMARY

Provided is a process for hydroconverting normal paraffins obtained from a renewable, bio originating source into lighter normal paraffins with minimal formation of iso-paraffins. In one embodiment, the process is a stable and efficient process for preparing ethylene in improved yields when starting with a bio feedstock. The bio feedstock will generally comprise triglycerides and fatty acids, plant derived oils and animal derived fats and oils. Such blends are often referred to as fats, oils and greases, or FOG.

The process first involves a hydrodeoxygenation reaction in which n-paraffins are made. The paraffins are then subjected to a hydroprocessing reaction under hydroconversion conditions. The paraffin containing feedstock generally comprises at least 3 wt. %, or in one embodiment, at least 5 wt. %, normal paraffins. The hydroconversion reaction is run in the presence of a specific type of zeolite-based catalyst, with the zeolite having a requisite topology and acid site density. The present zeolite is of a framework type with voids greater than 0.50 nm in diameter, which are accessible through apertures characterized by a longest diameter of less than 0.50 nm and a shortest diameter of more than 0.30 nm. In one embodiment, the zeolite comprises 10-1000 nm crystals. In one embodiment, the reaction is run in the presence of an LTA zeolite, and in another embodiment the LTA zeolite has an acid site concentration in the range of from 2.6 to 3.0 mol/l, and in one embodiment an acid site concentration of about 2.7 mol/l or greater. This acid site concentration has been found beneficial.

A chosen boiling range of n-paraffins is then collected from the hydroprocessing reactor. The range generally comprises C₂-C₆ n-paraffins. After conversion of the longer n-paraffins, any isoparaffins or naphthenics can then be separately subjected to destructive cracking to further increase their hydrogen content and thereby their suitability for steam cracking, if desired. Concentrating the n-paraffins in the C₂-C₆ carbon range and depleting the steam cracker feed from n-paraffins with a higher carbon number dramatically improves ethylene yields.

Among other factors the present process is stable and efficient in hydroconverting normal paraffins obtained from a renewable, bio source into lighter normal paraffins. It has been surprisingly found that the LTA zeolite used in the hydroconversion reaction, in one embodiment, with an acid site concentration around 2.7 mol/l, can reduce n-paraffins in high yield to n-paraffins in the ethane to naphtha boiling range of C₂-C₆ n-paraffins, and can do so continuously for months on end without losing its catalytic activity. The reaction is stable and does not exhibit any issues with feeds having extremely long n-paraffins, such as C₂₃⁺ in length. The C₂-C₆ n-paraffins can then be used as desired as building blocks for other chemicals.

Among other factors the present process is stable and efficient in providing a renewable feed that pyrolyzes into more ethylene. It has been surprisingly found that the LTA zeolite used in the hydroprocessing reaction, particularly with an acid site concentration around 2.7 mol/l, can reduce long n-paraffins in high yield to the n-paraffins in the ethane to naphtha boiling range of C₂-C₆ n-paraffins. Based on 3 months of operation without activity loss after line-out, current models indicate that a catalyst based on LTA-type zeolite would exhibit the typical run length of 2-4 years for base metal catalyst formulations and of 10-15 years for noble metal catalyst formulations at typical feeds and conditions. The catalyst sustainably hydrocracks extremely long n-paraffins, such as C₂₃⁺ in length. The stability of the hydrocracking process on the LTA-type zeolite catalyst is surprising. By providing such a suitable, renewable feedstock for a steam cracker, the preparation of ethylene and other lower olefins as chemical building blocks can be efficiently realized in good yield while using a steam cracker, while also employing a renewable bio source for the steam cracker feedstock.

### BRIEF DESCRIPTION OF THE DRAWINGS

FIG. 1 depicts a process for pyrolysis of a bio feedstock.

FIG. 2 depicts a process for upcycling biomass pyrolysis oil into chemical building blocks.

FIG. 3 depicts the composition of a C₁₂⁺ feed to hydroconversion.

FIG. 4 graphically depicts the results achieved from hydronormalizing a largely n-C₁₂⁺ feed.

FIG. 5 graphically depicts the change in product paraffin composition as a function of conversion of n-C₁₂⁺ paraffins.

FIG. 6 graphically depicts a steam cracker production.

### DETAILED DESCRIPTION

### Definitions

Steam cracking is a petrochemical process in which saturated hydrocarbons are broken down into smaller, often unsaturated, hydrocarbons. It is the principal industrial method for producing the lighter alkenes (or commonly olefins), including ethylene and propylene. Steam cracker units are facilities in which feedstocks such as ethane, propane, butane, liquefied petroleum gas (LPG), and naphtha are thermally cracked or pyrolyzed in the presence of steam by a short residence time in a furnace at high temperature to produce lighter hydrocarbons. After the cracking temperature has been reached, the gas is quickly quenched to stop the reaction in a transfer line heat exchanger or inside a quenching header using quench oil. The feed composition, the hydrocarbon-to-steam ratio, the cracking temperature and furnace residence time determine the product composition. Light hydrocarbon feeds such as ethane, LPGs, or light naphtha yield mostly lighter alkenes, including ethylene, propylene, and butadiene. Heavier hydrocarbon (full range and heavy naphtha or even heavier oil fractions) feeds yield some of these same products, but also aromatic pyrolysis oils.

Olefins such as ethylene, propylene and butylene are the raw materials of polyolefins. Polyolefins are one of the key building blocks of the global economy. Illustrating its critical roll to economic growth, steam cracking capacity tends to grow slightly ahead of global gross domestic product (GDP). Ethylene capacity in 2021 was 150,000 kiloton/annum (kta) and is growing at 6000-8000 kta of ethylene, produced in ever larger facilities. A current state-of-the-art cracker can produce 2000 kta of ethylene. At such large volumes feedstocks that make incrementally more ethylene have large financial benefits. Providing those feedstocks from renewable sources would render the ethylene renewable and even more beneficial.

A “naphtha” cracker is generally limited to feedstocks in the naphtha boiling range of 32-193° C. (C₄-C₁₀). An ethane cracker is generally limited to feeding ethane and some LPG. Most commercial liquid and gas steam crackers in current operation are naphtha and ethane crackers.

Hydroconversion and hydroconverting: A catalytic process which operates at pressures greater than atmospheric in the presence of hydrogen and which converts normal paraffins into lighter normal paraffins with a minimum of isomerization and without excessive formation of methane and ethane. This can also be referred to as hydro-normalization. Hydrotreating and hydrocracking are distinctly different catalytic processes but which also operate at pressures greater than atmospheric in the presence of hydrogen. Hydrocracking converts normal paraffins into lighter products comprising significant amounts of iso-paraffins. Hydrotreating does not convert significant quantities of the feedstock to lighter products but does remove impurities such as sulfur- and nitrogen-containing compounds. Also in comparative contrast, thermal cracking converts normal paraffins into lighter products with a minimum of branching, but this process does not use a catalyst, typically operates at much higher temperatures, forms more methane, and makes a mixture of olefins and normal paraffins.

A LTA (Linde Type A) zeolite is a zeolite that has voids greater than 0.50 nm in diameter, and apertures characterized by a longest diameter of less than 0.5 nm and a shortest diameter of more than 0.30 nm. Such LTA zeolites are described in the Atlas of Zeolite Structure Types, Fourth Revised Edition 1996.

An “aperture” in a zeolite is the narrowest passage through which an absorbing or desorbing molecule needs to pass to get into the zeolite's interior. The diameter of the aperture, dₐₚₚ (nm), is defined as the average of the shortest, dₛₕₒᵣₜ (nm), and the longest, d_{long} (nm) axis provided in the IZA (International Zeolite Association) Zeolite Atlas (http://www.iza-structure.org/databases/). Both normal- and iso-paraffins with a methyl group can pass through apertures with a d_{long}≥0.50 nm, but only normal-paraffins can pass through apertures with d_{long}<0.50 nm provided dₛₕₒᵣₜ>0.30 nm.

Apertures provide access to “voids”, the wider parts in the zeolite topology. The diameter of the void, d_{void} (nm), is characterized by the maximum diameter of a sphere that one can inflate inside such a void as per the IZA Zeolite Atlas (http://www.iza-structure.org/databases/). This characterizes, e.g., a fairly spherical LTA void (or cage) as one with a diameter of 1.1 nm, and an elongated AFX-type void as one with a spherical diameter of 0.78 nm. Voids are defined as cages if d_{void}/dₐₚₚ≥1.4 nm/nm. An LTA zeolite exhibits a topology with a defined combination of apertures and voids.

The term “bio” refers to biochemical and/or natural chemicals found in nature. Thus, a bio feedstock or bio oil would comprise such natural chemicals. The preferred starting bio feedstocks include triglycerides and fatty acids, plant-derived oils such as palm oil, canola oil, corn oil, and soybean oil, as well as animal-derived fats and oils such as tallow, lard, schmaltz (e.g., chicken fat), and fish oil, and mixtures of these. Such blends are often referred to as fats, oils and greases, or FOG. In one embodiment, the bio feedstock can comprise biomass pyrolysis oil prepared by pyrolyzing a bio feedstock material and hydrothermally liquefied oil prepared by decomposing the oil with water at high temperature and pressure (with either sub- or supercritical water).

In the first step of the present process a composition is produced from starting materials of biological origin. The process first comprises a hydrodeoxygenation (HDO) step for decomposing the structure of the biological ester or triglyceride constituent, and for removing oxygen, phosphorus and sulfur compounds, concurrently hydrogenating the olefinic bonds. The product is fractionated by distillation to give the desired fractions.

Biological raw materials from plants, animals or fish containing fatty acids and/or fatty acid esters may be used as the starting bio material. The raw material may be selected from vegetable oils, animal fats, fish oils, and mixtures thereof. Suitable biological raw materials include rapeseed oil, canola oil, colza oil, tall oil, sunflower oil, soybean oil, hemp oil, olive oil, linseed oil, mustard oil, palm oil, arachis oil, castor oil, coconut oil, animal fats such as suet, tallow, blubber, recycled alimentary fats, starting materials produced by genetic engineering, and biological starting materials produced by microbes such as algae and bacteria. Condensation products, esters, or other derivatives obtained from biological raw materials may also be used as starting materials.

In the hydrodeoxygenation (HDO) step, hydrogen gas and the biological constituent are passed to the HDO catalyst bed either in countercurrent or concurrent manner. In the HDO step, the pressure and the temperature range, for example, is between 20 and 150 bar, and between 200 and 500 degrees centigrade, respectively. In the HDO step, any suitable hydrodeoxygenation catalysts may be used.

Prior to the HDO step, the biological raw material may optionally be subjected to prehydrogenation under milder conditions to reduce or avoid side reactions of the double bonds.

After the HDO step, the long n-paraffins can be separated, or the HDO product stream comprising n-paraffins can be passed to the next step. The long n-paraffins, e.g., C₁₀-C₂₀ paraffins, are derived from a biological raw material. As noted above, the biological raw material can be originated from plants or animals. The material can be selected from vegetable oils, animal fats, fish oils and mixtures thereof. Examples of suitable biological raw materials include, but are not limited to, rapeseed oil, canola oil, colza oil, tall oil, sunflower oil, soybean oil, hemp oil, olive oil, linseed oil, mustard oil, palm oil, arachis oil, castor oil, coconut oil, animal fats such as suet, tallow, blubber, recycled alimentary fats, starting materials produced by genetic engineering, and biological starting materials produced by microbes such as algae and bacteria. Further condensation products, esters, or other derivates obtained from biological raw materials may also be used as starting materials. Also recycled raw materials of biological origin are suitable.

In one embodiment, the paraffins can be subjected to pyrolysis. The pyrolyzing is carried out by contacting the bio feedstock in a pyrolysis zone at pyrolysis conditions, where at least a portion of the feed(s) is cracked, thus forming a pyrolysis zone effluent comprising primarily olefins and paraffins. Pyrolysis conditions include a temperature of from about 400° C. to about 700° C., preferably from about 450° C. to about 650° C. Conventional pyrolysis technology teaches operating conditions of above-atmospheric pressures. See e.g., U.S. Pat. No. 4,642,401. Additionally, it has been discovered that by adjusting the pressure downward, the yield of a desired product can be controlled. See, e.g., U.S. Pat. No. 6,150,577. Accordingly, in some embodiments where such control is desired, the pyrolysis pressure is sub-atmospheric.

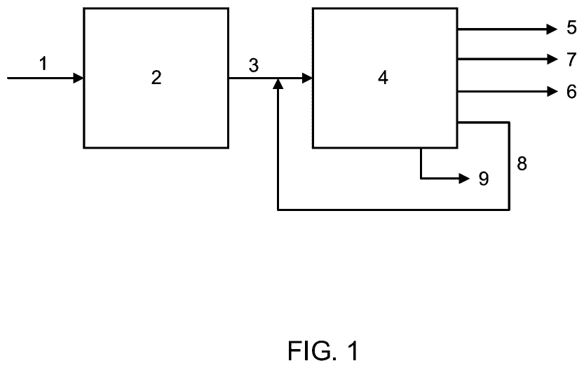

FIG. 1 shows a diagram of pyrolysis of a bio feedstock 1 that is hydrodeoxygenated at 2 in accordance with the present process. The deoxygenated bio feedstock 3 is converted in a pyrolysis unit 4 to offgas 5 and pyrolysis oil (liquid product) 6. The offgas 5 from the pyrolysis unit can be used as fuel to operate the pyrolysis unit. A distillation unit in the pyrolysis unit separates the pyrolysis oil to produce naphtha and diesel 7 products which can be sold to fuel markets, leaving a heavy pyrolysis oil fraction 8. The heavy pyrolysis oil fraction 8 can be recycled back to the pyrolysis unit 4 to maximize the fuel yield. Char 9 is removed from the pyrolysis unit 4. The heavy fraction 8 is rich in long chain, linear hydrocarbons, and is very waxy (i.e., forms paraffinic wax upon cooling to ambient temperature). Wax can be separated from the heavy fraction 8 and sold to the wax markets. The pyrolysis oil fraction 6 can then be forwarded/transported to a hydroconversion reactor.

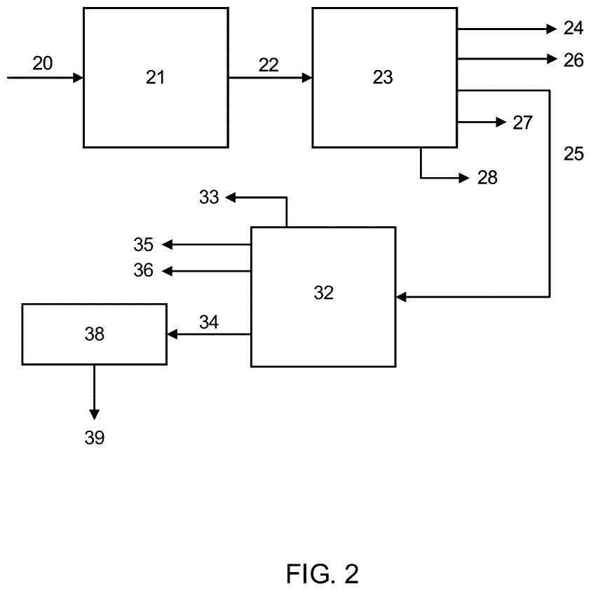

FIG. 2 shows the present integrated process in which pyrolysis, optionally with recycle, is integrated with hydro-normalization to produce a steam cracker feed. The product from the steam cracker may be used for effective polyethylene production. In FIG. 2, a bio feedstock 20 is hydrodeoxygenated in hydrodeoxygenation unit 21. The deoxygenated bio feedstock 22 is converted in pyrolysis unit 23 to offgas 24 and a pyrolysis oil 25 (liquid product) and optionally wax (solid product at ambient temperature). The offgas 24 from the pyrolysis unit can be used as fuel to operate the pyrolysis unit 23. The pyrolysis oil 25 may be separated, generally at an on-site distillation unit in the pyrolysis unit 23, into a naphtha/diesel fraction 26, and a heavy fraction 27. Char 28 is removed from the pyrolysis unit 23. Pyrolysis oil 25 from pyrolysis unit 23 is hydroconverted in hydroconversion reactor 32, producing offgas 33 and product stream 34 having an increased lighter (e.g., C₂ to C₆) n-paraffin content. Liquified petroleum gas (LPG) 35 and naphtha 36 streams may be separately produced from the hydroconversion. The n-paraffin product stream 34 is fed to steam cracker 38 and pyrolyzed into a steam cracker product comprising lower olefins 39 (e.g., ethylene).

The second step of the present process involves the hydro-normalization of the eventual feedstock to the naphtha steam cracker by hydroconverting normal paraffins into lighter normal paraffins with minimal formation of iso-paraffins. The process comprises hydroconverting a hydrocarbon feedstock comprising normal paraffins under hydroconversion conditions, in the presence of an LTA zeolite catalyst. The zeolite has voids greater than 0.50 nm in diameter, accessible through apertures characterized by a longest diameter of less than 0.50 nm and a shortest diameter of more than 0.30 nm. Most important, the present LTA zeolite exhibits an acid site concentration in the range of 2.6 to 3.0 mol/l, and most preferably about 2.7 mol/l. The present zeolite can be bound with alumina and extruded into a pellet or extrudate. The extrudate is loaded with 0.1 to 0.5 wt. % Pd, although any pellet or bound zeolite can be loaded with any hydrogenation function metal.

It is the zeolite-containing base into which the metal is loaded that is critical to the present processes. For it has been found that the present catalyst comprising a LTA zeolite in accordance herewith can provide the high conversion and minimal formation of iso-paraffins. It has been found that the key features of the catalyst zeolite include access to a pore system through apertures of a size less than 0.45 nm, and with the pore system containing voids greater than 0.50 nm in diameter. In another embodiment, the zeolite has voids greater than 0.50 nm in diameter, which are accessible through apertures characterized by a longest diameter of less than 0.5 nm and a shortest diameter of more than 0.30 nm. The LTA zeolite has such a zeolite framework. In one embodiment, the present LTA zeolite also exhibits an acid site concentration of from 2.6 to 3.0 mol/l, in one embodiment from 2.6 to 2.8 mol/l, and preferably about 2.7 mol/l. This is an LTA zeolite with a higher alumina concentration than normal.

Linde Type A, framework code LTA, is one of the most used zeolites in separations, adsorption, and ion exchange. This structure contains large spherical cages (diameter ˜11.4 Å) that are connected in three dimensions by small 8-membered ring (8MR) apertures with a diameter of 4.1 Å. LTA is normally synthesized in hydroxide media in the presence of sodium with Si/Al ˜1 mol/mol. By changing the cation, the limiting diameter of the 8MR apertures can be tuned, creating the highly used series of adsorbents 3A (potassium form, 2.9 Å diameter), 4A (sodium form, 3.8 Å diameter) and 5A (calcium form, 4.4 Å diameter) that are used to selectively remove species such as water, NH₃, SO₂, CO₂, H₂S, C₂H₄, C₂H₆, C₃H₆ and other n-paraffins from gases and liquids. Detergents deploy zeolite 4A because it softens water by replacing calcium and magnesium ions in “hard” water with sodium ions. While LTA zeolites are used in vast quantities for the aforementioned applications, the industry has considered the low framework Si/Al ratio and subsequent poor hydrothermal stability limits as limiting factors to succeeding under more demanding process conditions that are commonly found in catalytic applications. Yet surprisingly, the present process is found to be stable and efficient using an LTA zeolite with an acid site concentration of from 2.6 to 3.0 mol/l.

The stability of the present LTA zeolites with 0.4 nm wide constrictions in hydroconverting n-alkanes longer than n-hexane (n-C₆) is stunning. Based on 3 months of operation without activity loss after line-out, current models indicate that a catalyst based on LTA-type zeolite would exhibit the typical run length of 2-4 years for base metal catalyst formulations and of 10-15 years for noble metal catalyst formulations at typical feeds and conditions. The catalyst sustainably hydrocracks extremely long n-paraffins, such as C₂₃⁺ in length. The stability of the hydrocracking process on the LTA-type zeolite catalyst is surprising because it is well-established that a (de) hydrogenation function needs to activate n-paraffins into n-olefins, and that these n-olefins need to enter ˜11 nm wide LTA-type cages before isomerizing into iso-olefins (see J. E. Schmidt et al, ACS Catalysis vol. 13, 2023 pp 6710-6720). These iso-olefins are trapped inside the LTA-type cages, for they are too large to egress through the ˜5 nm wide LTA-type windows (see P. B. Weisz, V. J. Frilette, J. Phys. Chem. vol. 64, 1960, p 382). Well-established mechanisms explain how iso-olefins crack into mixtures of iso-paraffins, iso-olefins, n-paraffins and n-olefins (J. Weitkamp, P. A. Jacobs, J. A. Martens, Appl. Catal. vol. 8, 1983, pp. 123-141). An iso-paraffin would require activation into an iso-olefin to enable isomerization into an n-olefin and escape from the LTA-type cage. Without a noble metal function iso-paraffins would accumulate inside the LTA-type cages, blocking access to the zeolite and deactivating the catalyst. Surprisingly, this deactivation was not observed, so that the catalyst sustainably converted longer n-paraffins into desirable linear paraffins in the C₂-C₆ carbon number range.

The discovered stability of the present LTA zeolites with 0.4 nm wide constrictions that hydrocrack n-C₁₂⁺ and longer n-alkanes out of feed stocks containing such n-paraffins for at least three months is not intuitive. It is not intuitive because n-C₁₂⁺ and longer n-alkanes inherently hydrocrack into branched alkanes. This would imply that the primary branched alkene and alkane products would have further isomerized into n-alkenes so as to egress through 0.4 nm wide constrictors. Particularly for i-butanes (that are allegedly primary cracking products) it is not clear what mechanism would be involved to let them egress.

The discovered hydroprocessing stability of LTA zeolites with an acid concentration as high as 2.7 mol/l is truly a surprise. Previously, it has been shown that the stability is inversely proportional to acid concentration, and the longheld belief in the industry is that stable operation requires an acid concentration of at most 1.8 mol/l. To improve the stability of LTA zeolites with a high acid site concentration, methods were developed to place the metals inside the zeolite to little avail. At acid concentrations higher than 1.8 mol/l, catalysts are supposed to coke up or crumble.

The stable operation of the present LTA zeolite with an acid concentration as high as 2.7 mol/l (well above the historically suggested 1.8 mol/l threshold) in the hydro-normalization of n-alkanes as long as n-C₁₂⁺ remains a bit of a mystery and surprise. The present LTA zeolite having the requisite acid site concentration can continue in operation for at least 3 months and even longer, e.g., 6 months to two years or even 5 years or longer. This is counter intuitive, yet this is what has been discovered.

The catalyst based on the present LTA zeolite can typically contain a catalytically active hydrogenation metal. The presence of a catalytically active hydrogenation metal leads to product improvement, especially IV and stability. Typical catalytically active hydrogenation metals include chromium, molybdenum, nickel, vanadium, cobalt, tungsten, zinc, platinum, and palladium. The metals platinum and palladium are especially preferred, with platinum most especially preferred. If platinum and/or palladium is used, the total amount of active hydrogenation metal is typically in the range of 0.1 wt. % to 5 wt. % of the total catalyst, usually from 0.1 wt. % to 2 wt. %.

The zeolite can be loaded with a hydrogenation function metal or a mixture of such metals either as is or bound with a suitable binder, such as silica, alumina, or titania. Such hydrogenating metals are known in the art and have been discussed generally earlier. The preferred metal is typically either a noble metal, such as Pd, Pt, and Au, or a base metal, such as Ni, Mo and W. A mixture of the metals and their sulfides can be used. The loading of the zeolite with the metals can be accomplished by techniques known in the art, such as impregnation or ion exchange. The hydrogenation function metal is loaded on such a selected zeolite to create the catalyst. The created catalyst can then be used in the hydroconversion process.

The feedstock for the second step of the process, hydroconversion, is generally a hydrocarbon feedstock which comprises at least 5 wt. % normal paraffins. The feedstock of normal paraffins is obtained from the products of the HDO step. Greater benefit is achieved when the hydrocarbon feedstock comprises at least 20 wt. %, even better when at least 50 wt. % normal paraffins, and in particular at least 80 wt. % normal paraffins. The normal paraffins in the feedstocks of the present process comprise large amounts of C₇⁺ normal paraffins, or in one embodiment C₁₀⁺, or in one embodiment C₁₂⁺ n-paraffins. The amount of C₇⁺, or C₁₀⁺, or C₁₂⁺ n-paraffins based on the total weight of the hydrocarbon feedstock is generally at least 5 wt. %, but more likely at least 10 wt. %, or in one embodiment at least 20 wt. %. Such feedstocks can be obtained from a wide variety of bio sources.

In an embodiment, the feedstock's aromatics and organic nitrogen, sulfur, oxygen, halogen and silicon content is reduced. This can be achieved by hydrotreating the feedstock prior to the hydroconversion, if needed. Contacting the feedstock with a hydrotreating catalyst may serve to effectively hydrogenate aromatics in the feedstock and to remove N-, S-, O-, Cl-, Br-, F-, Si-containing compounds from the feed.

The conditions under which the second step of the present processes, i.e., the hydro-normalization reaction or hydroconversion reaction, is carried out will generally include a temperature within a range from about 390° F. to about 800° F. (199° C. to 427° C.). In an embodiment, the temperature is in the range from about 550° F. to about 700° F. (288° C. to 371° C.). In a further embodiment, the temperature may be in the range from about 590° F. to about 675° F. (310° C. to 357° C.). The pressure may be in the range from about 50 to about 5000 psig, and typically in the range from about 100 to about 2000 psig.

The normal paraffin-rich product recovered from the hydroconversion can then be used as desired. For example, as a solvent in the paint industry, as a new material in making chemicals, for further processing into fuels or even gasoline. One important use is as a feed to a steam cracker, as discussed. Using the present LTA zeolite-based catalyst with the higher acid site concentration provides a C₂-C₆ product in improved yields, which product can be collected from the hydroconversion process.

The product enriched in lower normal paraffins (particularly C₂-C₆ linear paraffins) recovered from the hydroconversion can then be passed to a steam cracker. Using the present LTA zeolite-based catalyst which combines a higher acid site concentration with a smaller (<100 nm) crystal size concentrates the C₂-C₆ linear paraffin fraction in the hydroprocessing products at the expense of the fraction of longer (C₇⁺) n-alkanes. The process enriches the product stream by at least 2 wt. %, more preferably at least 3 wt. %, and in one embodiment at least 4 wt. % C₂-C₆ n-paraffins. The C₂-C₆ product stream can be split into streams comprising C₂-C₃ and C₄-C₆ n paraffins, or the C₂-C₆ n-paraffins can be used as a single stream.

The third step is a steam cracking step in a steam cracker. The steam cracking process is known in the art. Steam cracking a hydrocarbon feedstock produces olefin streams containing olefins such as ethylene, propylene, and butenes as well as aromatics. The present hydroconversion process provides an excellent feedstock for a steam cracker because it increases the ethylene yield at the cost of propylene and aromatics yields.

In steam cracking, a gaseous or liquid hydrocarbon feed like ethane, LPG, light or full-range naphtha is diluted with steam and heated in a furnace in the absence of oxygen. Typically, the reaction temperature is between 80° and 900° C. and the residence time is on the order of milliseconds. The pyrolyzing gases flow at close to the speed of sound. After pyrolysis, the gas is quickly quenched to stop the reaction in a transfer line heat exchanger or inside a quenching header using quench oil.

The products produced in the reaction depend on the composition of the feed and on the conditions, such as the hydrocarbon-to-steam ratio, the cracking temperature and furnace residence time. Light hydrocarbon feeds (ethane, LPG, light naphtha) yield lighter alkenes, including ethylene, propylene, and butadiene. Heavier hydrocarbon feeds (full range and heavy naphtha, hydrogenated gas oils) also yield these lighter products as well as aromatic pyrolysis oils suitable for aromatics extraction and for use in the production of needle coke or synthetic graphite. The present process is directed to preparing lower alkenes, and in particular ethylene and at minimizing pyrolysis oil make. The C₂-C₆ light naphtha range prepared by the first step hydroconversion allows for high yields of lower alkenes such as ethylene. The impact of increased ethylene production by the present process is dramatic. The dollar value in the larger yield of desirable products (olefins and BTX) and the lower feed rate and the lower associated energy consumption is massive.

In one embodiment, the steam cracking step is specifically an ethane steam cracking step. The ethane steam cracking process is known in the art. An ethane cracking process cracks the ethane into ethylene. Ethylene is the desired product, although other lower olefins are useful. But it is ethylene which is most desired. The present hydroconversion process using the present LTA zeolite provides an excellent feedstock for an ethane steam cracker because it increases the ethylene yield at the cost of propylene and aromatics yields. The present hydroconversion process increases the ethylene plus butadiene yield at least 4 wt. %, and in one embodiment, at least 8 wt. %, while the steam cracker is run at high severity. The present hydroconversion process also results in an increase of ethylene plus butadiene by at least 3 wt. %, and in one embodiment, at least 6 wt. %, while the steam cracker is run at low severity.

Ethane crackers are typically designed to only crack C₂ and C₃ n-paraffins. Thus, if the C₂-C₆ product stream is split, only the C₂-C₃ portion can be fed to an ethane cracker. However, if the heater in the ethane cracker is revamped with appropriate piping, which is known, all of the C₂-C₆ can be cracked in the ethane cracker.

In an ethane steam cracking process, a gaseous or liquid hydrocarbon feed like ethane, LPG, light or full-range naphtha is diluted with steam and heated in a furnace in the absence of oxygen. Typically, the reaction temperature is between 80° and 900° C. and the residence time is on the order of milliseconds. The pyrolyzing gases flow at close to the speed of sound. After pyrolysis, the gas is quickly quenched to stop the reaction in a transfer line heat exchanger or inside a quenching header using quench oil.

If ethylene polymerization is desired, the process of FIG. 2 depicts the production of a steam cracker product that may be used to produce polyethylene. For example, ethylene 39 from the steam cracker 38 can be recovered and used as a chemical or passed to ethylene polymerization to make polyethylene and polyethylene products.

The following examples are provided in order to further illustrate the present process. However, the examples are not meant to be limiting.

### Example 1

### Example of LTA Zeolite Based Noble Metal Catalyst.

A catalyst was made by extruding 50 wt. % LTA zeolite with 50-wt-% alumina (Pural TH80 from Sasol). The KAQ-type extrudates were loaded with 0.5 wt. % Pd. The LTA zeolite used had an acid site concentration of about 2.7 mol/l.

### Example 2

A process to selectively hydro-normalize the long n-paraffins in a feedstock into particularly C₂-C₆ n-paraffins has been discovered. The process thereby provides a tailored feedstock for a naphtha steam cracker, which selectively pyrolyzes the C₂-C₆ n-paraffins to monetize these hydro-normalized products.

Hydro-normalization is accomplished through hydroprocessing a paraffin feedstock. The paraffin feedstock is obtained by hydrodeoxygeneating a bio feedstock and collecting n-paraffins. The hydroprocessing catalyst can be the LTA zeolite described in Example 1, comprising a LTA zeolite with an optimum acid site concentration around 2.7 mol/l. Key features of the hydro-normalization process are that the catalyst selectively converts longer normal paraffins in the feed into C₂-C₆ n-paraffins. Since the feed in the example has an initial boiling point of 390° F., the shortest n-paraffin in feed is an n-dodecane (or n-C₁₂ with a 421° F. boiling point, whereas n-undecane or n-C₁₁ exhibits a 385° F. boiling point).

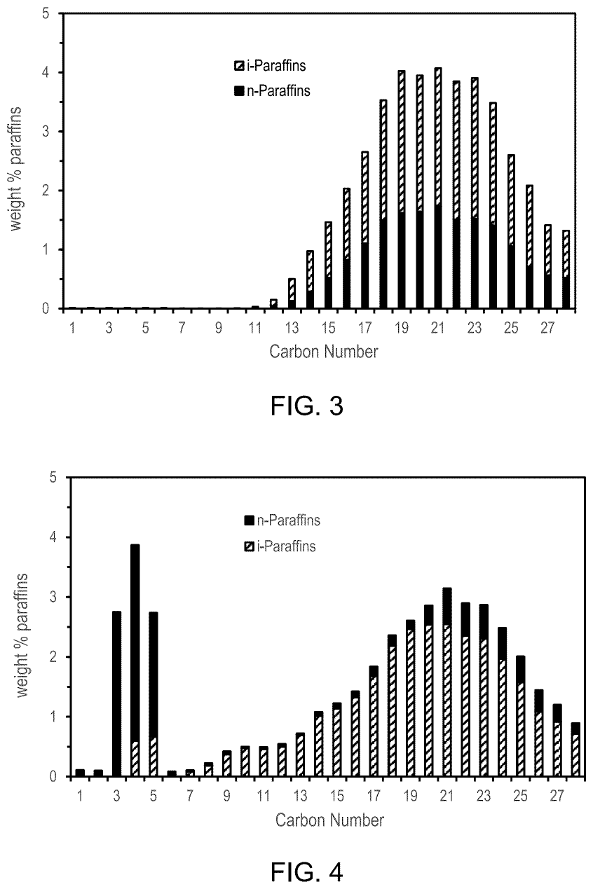

See FIG. 3 which shows a feed exclusively containing n-C₁₂^{plus} with negligible n-C₁₂^{minus}. The products at 81% n-C₁₂^{plus} conversion are nearly exclusively C₂-C₅ n-paraffins. See FIG. 4.

Contrary to the expectations of the art, the present LTA zeolites have been surprisingly found to not exhibit a dramatic preference for processing n-paraffins up to the length that could easily fit inside a single LTA cage, which would limit the length of readily hydrocracked n-paraffins to that of n-tricosane or n-C₂₃ (716° F. boiling point) Longer n-paraffins (n-triaocontane or n-C₃₀, 840° F. boiling point and longer) are still successfully processed. In addition to a remarkable selectivity for hydrocracking selectively n-C₁₂ and longer n-alkanes, the catalyst also makes some minimal i-paraffins.

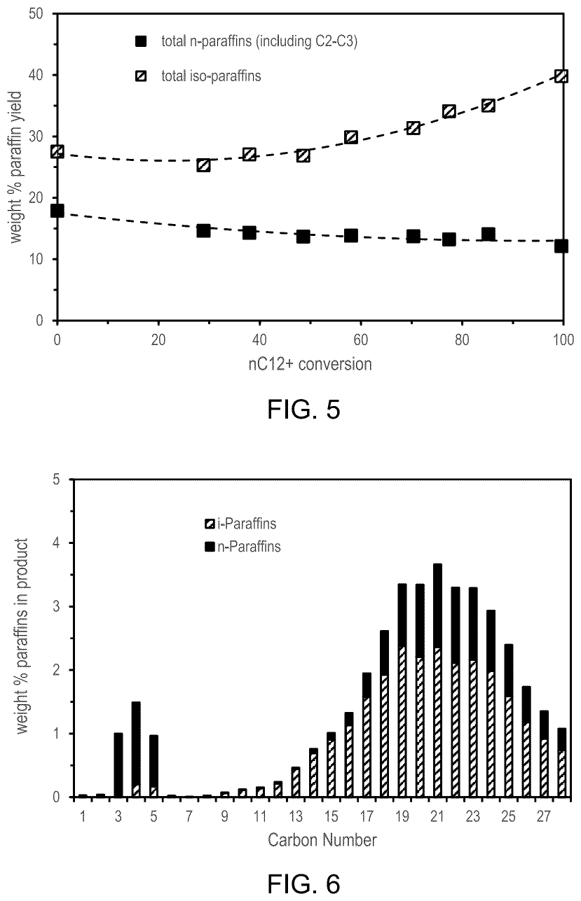

For the specific feed used, i-paraffin production picks up above 50% n-C₁₂^{plus} conversion. See FIG. 5. The increase in i-paraffin fraction (and a concomitant decrease in the n-paraffin fraction) signals a likely increase in the total paraffin yield through dealkylation and ring opening.

### Example 3

### Example of Steam Cracker Feed Properties.

A hydrodeoxygenated feedstock can be hydroprocessed into an improved steam cracker feed as described in Table 1 below at 1550 psia H₂, 1.6 LHSV, 5100 scf/b H₂. A steam cracker feed produced at 60% n-C₁₂⁺ conversion in the present hydro-normalization step is characterized in Table 1 as follows:

TABLE 1

| | Feed | Hydroprocessed |
|---|---|---|
| API (°) | 35.4 | not measured |
| S (ppm-wt) | 5.3 | <0.3 |
| N (ppm-wt) | <0.3 | <0.3 |
| wt-% paraffins (GC × GC) | 45 | 52 |
| wt-% n-C₁₂⁺ | 18 | 0 |
| wt-% C₂-n-C₆ | 0 | 12 |
| Composition | Wt % | Wt % |
| Methane | 0.00 | 0.03 |
| Ethane | 0.00 | 0.04 |
| Propane | 0.00 | 1 |
| i-Butane | 0.00 | 0.2 |
| n-Butane | 0.00 | 1.29 |
| C4− | 0.00 | 2.56 |
| C5− 180′F. | 0.00 | 2.2 |
| 180-350′F. | 0.00 | 0.59 |
| 350-500′F. | 7.2 | 9.58 |
| 500′F.+ | 92.8 | 86.13 |
| C5+ | 100.0 | 98.5 |
| WLP Dist. By Wt % | | |
| 0.5/5% | 392/478 | 95/445 |
| 10/30% | 519/606 | 495/598 |
| 50% | 666/ | 660/ |
| 70/90% | 719/799 | 716/796 |
| 95/99.5% | 842/955 | 840/956 |

The distribution between n- and iso-paraffins as determined by GC×GC is as shown in FIG. 5.

When switching from a feed without hydro-normalization to one with hydro-normalization, a dramatically lower feed rate (ton/hr) is needed to reach the same desirable ethylene (C₂H₄) yield. At this feed rate, the hydronormalized feed exhibits a shift toward desirable olefins (C₂H₄, C₃H₆, C₄H₈) and aromatics (benzene, toluene, xylenes or BTX) at the cost of heavier products (particularly pyrolysis fuel oil or PFO) (see Table 2 below). This amounts to a dramatic increase in carbon and energy efficiency of the steam cracking process.

TABLE 2

| | Original Feed | | Hydroprocessed | |
|---|---|---|---|---|
| | High Sev | Low Sev | High Sev | Low Sev |
| Feed, T/h | 324.1 | 392.7 | 307.7 | 379.1 |
| H2 | 2.4 | 2.2 | 2.4 | 2.1 |
| CH4 | 37.2 | 34.2 | 37.1 | 33.6 |
| C2H4 | 100 | 100 | 100 | 100 |
| C3H6 | 46.1 | 62.2 | 41.9 | 61.3 |
| C4H6 | 18.2 | 21.9 | 17.8 | 21.7 |
| C4H8 | 11.4 | 24 | 9.8 | 23.8 |
| C4H10 | 0.4 | 0.6 | 0.4 | 0.6 |
| C5-400 F. PGO | 61.4 | 77.2 | 58.1 | 76.3 |
| 400 F. + PFO | 47 | 70.4 | 40.4 | 59.8 |
| Selectivity, w/w | | | | |
| C2H4 | 30.86 | 25.46 | 32.5 | 26.38 |
| C2H4 + C3H6 | 45.07 | 41.3 | 46.12 | 42.53 |
| C2H4 + C3H6 + C4H8 | 50.69 | 46.87 | 51.9 | 48.27 |
| BTX | 12.08 | 7.44 | 12.6 | 7.45 |

Switching from an as-is feed where the n-paraffins reside in the 350° F.⁺ boiling range to a feed where some 60% of the n-paraffins have been transferred to the front end of the boiling range (−128° F. to 156° F.) as quantified the by n-C₁₂⁺ conversion (FIG. 3), increases the yield in valuable C₂ olefins, hydrogen gas, lowers the yield of lower-value byproducts such as pyrolysis gasoline (PGO) and pyrolysis fuel oil (PFO), residues and coke, prolongs operating cycles, lower operating severity (Table 3), and lower utilities, CO₂ production and CAPEX per kiloton/annum (kta) of olefins produced. (See Table 2 above). This amounts to a dramatic increase in carbon, energy and capital efficiency in the steam cracking process (FIG. 6).

TABLE 3

| | |
|---|---|
| High Severity | Low Severity |

| | Orig. | Hydr. | Orig. | Hydr. |
|---|---|---|---|---|
| | Feed | Feed | Feed | Feed |
| Feed, kta | 5800 | 5800 | 7000 | 7000 |
| hydrogen | 43 | 45 | 39 | 39 |
| methane | 666 | 699 | 610 | 620 |
| ethylene | 1790 | 1885 | 1783 | 1846 |
| propylene | 825 | 790 | 1109 | 1132 |
| butadiene | 326 | 336 | 390 | 401 |
| butenes | 204 | 185 | 428 | 439 |
| butane | 7 | 8 | 11 | 11 |
| C5-400 F. PGO | 1099 | 1095 | 1376 | 1409 |
| 400 F. + PFO | 841 | 762 | 1255 | 1104 |
| Selectivity, w/w | 31 | 33 | 25 | 26 |
| C₂H₄ | 45 | 46 | 41 | 43 |
| C₂H₄ + C₃H₆ | 51 | 52 | 47 | 48 |
| C₂H₄ + C₃H₆ + C₄H₈ | 12 | 13 | 7 | 7 |
| BTX | 31 | 33 | 25 | 26 |

According to the above yield structure (Table 3), a state-of-the-art steam cracker that feeds 5800 kta of the feeds described in Table 1 produces about 1800 kta ethylene. The hydroprocessed feed produces 95 kta more ethylene at the cost of mostly (80 kta) low-value PFO (FIG. 6). At the current ethylene prices of 1000-1100 $/ton this increase in ethylene make amounts to a ˜100 MM$/annum increase in steam cracker value creation.

As used in this disclosure the word “comprises” or “comprising” is intended as an open-ended transition meaning the inclusion of the named elements, but not necessarily excluding other unnamed elements. The phrase “consists essentially of” or “consisting essentially of” is intended to mean the exclusion of other elements of any essential significance to the composition. The phrase “consisting of” or “consists of” is intended as a transition meaning the exclusion of all but the recited elements except for only minor traces of impurities.

As those skilled in the art will appreciate, numerous modifications and variations of the present invention are possible considering these teachings, and all such are contemplated hereby. For example, in addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

All of the publications cited in this disclosure are incorporated by reference herein in their entireties for all purposes.

What is claimed is:

1. A process for pyrolyzing a renewable feedstock comprising C₂-C₆ n-paraffins in a steam cracker, comprising:
a) subjecting a bio feedstock to hydrodeoxygenation to prepare n-paraffins, and optionally hydrotreatment;
b) subjecting the n-paraffins to a hydroconversion reaction under hydroconversion conditions in the presence of a catalyst that converts n-C₇⁺ paraffins into linear paraffins in the C₂-C₆ range; and
c) collecting a stream with an increased C₂-C₆ n-paraffin concentration at the expense of the n-C₇+ concentration from the hydroconversion reaction and pyrolyzing same in a steam cracker into lower olefins.

2. The process of claim 1, wherein the process can run for at least 3 months, or at least 6 months, or at least 2 years, or at least 5 years without changing the catalyst or regenerating the LTA-type zeolite-based catalyst.

3. The process of claim 1, wherein the C₂-C₆ linear paraffin concentration increases the ethylene yield on a feed basis by at least 3%, or by at least 4%, or by at least 5%.

4. The process of claim 1, wherein the catalyst comprises an LTA-type zeolite having an acid site concentration of about 2.6 to 3.0 mol/l or about 2.7 mol/l.

5. The process of claim 1, wherein the n-paraffins in b) comprises at least 5 wt. %, or at least 10 wt. % C₇⁺ normal paraffins.

6. The process of claim 1, wherein the catalyst comprises an LTA zeolite, optionally, shaped with a binder into a pellet, wherein the catalyst is loaded with a hydrogenation function metal, wherein the hydrogenation function metal comprises a noble metal; or the hydrogenation function metal comprises Pd, Pt, Re, Ru, Sn, Au or a mixture thereof; or the hydrogenation function metal comprises Ni, Co, Mo, W, their sulfides, or a mixture thereof; or wherein the LTA zeolite comprises mixed metal sulfides.

7. The process of claim 1, wherein the per-pass conversion of the normal paraffins in the feedstock is between 25 and 99%.

8. A process for increasing the steam cracker ethylene yield at the cost of the pyrolysis fuel oil yield by hydrocracking long n-paraffins (C₁₀⁺) into lighter C₂-C₆ n-paraffins, comprising:
a) subjecting a bio feedstock to a hydrodeoxygenation to make n-paraffins;
b) subjecting the n-paraffins to a hydroconversion reaction under hydroconversion conditions in the presence of an LTA zeolite-based catalyst comprising of 10-1000 nm crystals with an acid site concentration in the range of from 2.6 to 3.0 mol/l; and
c) forwarding a product stream enriched in C₂-C₆ n-paraffins and depleted in longer n-paraffins by the prior hydroconversion reaction to a steam cracker for pyrolysis into ethylene.

9. The process of claim 8, wherein a fraction in the −128° F. to 400° F. (−89° C. to 204° C.) boiling range is collected from the hydroprocessed product stream in b) is passed to a naphtha cracker and/or wherein the fraction in the −128° F. to 156° F. (−89° C. to 69° C.) boiling range collected from the hydroprocessed product stream in b) is passed to a gas cracker.

10. The process of claim 8, wherein the hydroconversion process in b) enriches the hydroprocessed product stream by at least 2 wt. %, or by at least 3 wt. % or by at least 4 wt. % in C₂-C₆ n-paraffins; and/or wherein the hydroconversion process in b) converts at least 10% or at least 20% or at least 40% of the n-paraffins in the bio feedstock into mostly C₂-C₆ n-paraffins.

11. The process of claim 8, wherein the hydroconversion increases the ethylene plus butadiene yield by at least 4 wt. % or at least 8 wt. % when the steam cracker is run at high severity and/or wherein the hydroconversion increases the ethylene plus butadiene yield by at least 3 wt. % or at least 6 wt. % when the steam cracker is run at low severity.

12. The process of claim 8, wherein hydroconversion decreases the undesirable pyrolysis fuel oil (“PFO”) yield by at least 5 wt. % or at least 10 wt. % when the steam cracker is run at high severity and/or wherein hydroconversion decreases the undesirable pyrolysis fuel oil (“PFO”) yield by at least 6 wt. % or at least 12 wt. % when the steam cracker is run at low severity.

13. The process of claim 8, wherein the hydroconversion increases the ethylene yield by at least 4 wt. %; or wherein the hydroconversion increases the yield of valuable chemicals (viz. ethylene, propylene, butadiene and pyrolysis gasoline (“PGO”)) by at least 4 wt. %, or by at least 10 wt. %.

14. The process of claim 8 wherein the LTA zeolite is loaded with a hydrogenation function, wherein the hydrogenation function metal comprises a noble metal; or the hydrogenation function metal comprises Pd, Pt, Re, Ru, Sn, Au or a mixture thereof; or wherein the hydrogenation function metal comprises Ni, Co, Mo, W, their sulfides, or a mixture thereof.

15. The process of claim 14, wherein the per-pass conversion of the normal paraffins in the feedstock in a hydroconversion recycle operation is between 10 and 99%.

16. The process of claim 8, wherein the feedstock in b) comprises at least 5 wt. %, or at least 10 wt. % C₇⁺ normal paraffins.

17. The process of claim 8, wherein the product stream from the hydroprocessing in b) passes on to a mixed-feed steam cracker.

18. The process of claim 8, wherein a hydroconversion process increases the amount of iso-paraffins in the product compared to the feed by more than 5 wt. % but less than 50 wt. %.

19. A continuous process for converting a bio feedstock into chemical building blocks via pyrolysis comprising:
(a) selecting a bio feedstock;
(b) passing the bio feedstock through a pyrolysis reactor to produce a pyrolyzed effluent;
(c) separating the pyrolyzed effluent into off-gas, char, and a pyrolysis bio-oil, optionally, wherein at least some contaminants are removed from the recovered pyrolysis bio-oil;
(d) subjecting the pyrolysis bio-oil to hydrodeoxygenation;
(e) passing the hydrodeoxygenated pyrolysis bio-oil from (d) to a hydroconversion reactor comprising a hydroconversion catalyst based on a LTA-type zeolite having an acid site concentration in the range of 2.6 mol/l to 3.0 mol/l or 2.6 mol/l to 2.8 mol/l or about 2.7 mol/l;
(f) recovering hydrocarbons from the hydroconversion reactor; and
(g) passing the hydrocarbons from (f) to a steam cracker for olefin and aromatics production.

20. A process comprising:
subjecting a bio feedstock to hydrodeoxygenation to make n-paraffins, optionally, wherein the bio feedstock is subjected to hydrotreatment;
recovering a hydrocarbon product stream comprising at least 5 wt. % normal paraffins; and
subjecting the hydrocarbon stream comprising at least 5 wt. % or at least 10 wt. % normal paraffins to a hydroconversion reaction under hydroconversion conditions in the presence of a LTA-type zeolite-based catalyst, the zeolite having an acid site concentration in the range of from 2.6 to 3.0 mol/l or 2.6 to 2.8 mol/l or about 2.7 mol/l, optionally, at a 10-1000 nm crystal size, to produce a product comprising C₂-C₆ n-paraffins.

21. The process of claim 20, wherein the process may be run for at least 3 months, or at least 6 months, or at least 2 years, or at least 5 years without changing the LTA catalyst or regenerating the LTA catalyst.

22. The process of claim 20, wherein the LTA zeolite comprises a binder, such as an alumina, and the catalyst is loaded with a hydrogenation function metal, such as a noble metal, for example, Pd, Pt, Re, Ru, Sn, Au or a mixture thereof; and/or Ni, Co, Mo, W, their sulfides, or a mixture thereof; and/or mixed transition metal sulfides.

23. The process of claim 20, wherein the per-pass conversion of the normal paraffins in the feedstock is between 20 and 99% and/or wherein the product stream is further converted to chemicals or used in the preparation of fuels or gasoline.
